(19) United States
(12) Patent Application Publication
(10) Pub. No.: US 2025/0387042 A1
(43) Pub. Date: Dec. 25, 2025

(54) AUSCULTATION SYSTEM

(71) Applicant: Murata Manufacturing Co., Ltd., Nagaokakyo-shi (JP)

(72) Inventor: Hirofumi TSUCHIMOTO, Nagaokakyo-shi (JP)

(21) Appl. No.: 19/228,845

(22) Filed: Jun. 5, 2025

(30) Foreign Application Priority Data
Jun. 21, 2024 (JP) 2024-100766

(51) Int. Cl. A61B 5/08; A61B 5/00; A61B 7/00; A61B 7/04

(52) U.S. Cl. CPC A61B 5/0803; A61B 5/7264; A61B 5/7405; A61B 5/742; A61B 7/003; A61B 7/04; A61B 2560/0462; A61B 2562/0204

(57) ABSTRACT

An auscultation system includes a body sound acquisition unit that obtains a body sound, a feature extraction circuit that extracts a feature signal representing the feature of the body sound, a sound source that outputs a predetermined sound source signal, a mixer circuit that mixes the feature signal with the sound source signal, and an output unit that outputs, for visual display, at least one of a signal outputted from the mixer circuit or information that is based on the signal.

### CROSS REFERENCE TO RELATED APPLICATION

This application claims priority from Japanese Patent Application No. 2024-100766, filed on Jun. 21, 2024. The content of these applications are incorporated herein by reference in its entirety.

### BACKGROUND OF THE DISCLOSURE

### 1. Field of the Disclosure

The present disclosure relates to an auscultation system.

### 2. Description of the Related Art

Japanese Unexamined Patent Application Publication No. 2000-314729 discloses a stethoscope that uses ultrasonic waves for various diagnoses. The stethoscope disclosed in Japanese Unexamined Patent Application Publication No. 2000-314729 includes a function for converting ultrasonic waves for examination into audible sound waves and makes it possible to directly auscultate waveform characteristics of ultrasonic waves generated by, for example, living bodies and machines.

### BRIEF SUMMARY OF THE DISCLOSURE

The present disclosure provides an auscultation system that makes it easier for a user to recognize body sounds of a subject.

An auscultation system according to an aspect of the present disclosure includes a body sound acquisition unit that obtains a body sound; a feature extraction circuit that extracts, from the body sound, a feature signal representing the feature of the body sound; a sound source that outputs a predetermined sound source signal; a mixer circuit that mixes the feature signal with the sound source signal; and an output unit that outputs, for visual display, at least one of a signal outputted from the mixer circuit and information that is based on the signal.

An auscultation system according to another aspect of the present disclosure includes a body sound acquisition unit that obtains a body sound; a feature extraction circuit that extracts a feature signal from the body sound; a sound source that outputs a predetermined sound source signal; a modulation circuit that modulates the sound source signal by using the feature signal; and an output unit that outputs at least one of a signal outputted from the modulation circuit and information that is based on the signal.

The present disclosure makes it easier for a user to recognize body sounds of a subject.

### BRIEF DESCRIPTION OF THE SEVERAL VIEWS OF THE DRAWINGS

FIG. 1 is a schematic diagram illustrating an example of an auscultation device according to a first embodiment;

FIG. 2 is a block diagram illustrating an example of a functional configuration of the auscultation device according to the first embodiment;

FIG. 3 is a flowchart illustrating an example of an operation of the auscultation device according to the first embodiment;

FIG. 4 is a schematic diagram illustrating an example of a time-amplitude graph displayed on a display monitor in the first embodiment;

FIG. 5 is a schematic diagram illustrating an example of a time-amplitude graph displayed on the display monitor in the first embodiment;

FIG. 6 is a schematic diagram illustrating an example of a spectrogram image displayed on the display monitor in the first embodiment;

FIG. 7 is a block diagram illustrating an example of a configuration of an auscultation device according to a second embodiment;

FIG. 8 is a flowchart illustrating an example of an operation of the auscultation device according to the second embodiment;

FIG. 9 is a schematic diagram illustrating an example of a spectrum displayed on a display monitor in the second embodiment;

FIG. 10 is a schematic diagram illustrating an example of a spectrum displayed on the display monitor in the second embodiment;

FIG. 11 is a block diagram illustrating an example of a configuration of an auscultation device according to a third embodiment;

FIG. 12 is a flowchart illustrating an example of an operation of the auscultation device according to the third embodiment;

FIG. 13 is a graph for describing an example of a method of calculating a risk value;

FIG. 14 is a block diagram illustrating an example of a configuration of an auscultation system according to a fourth embodiment; and

FIG. 15 is a block diagram illustrating an example of a configuration of an auscultation device according to a second variation.

### DETAILED DESCRIPTION OF THE DISCLOSURE

### 1. First Embodiment

### 1-1. Configuration

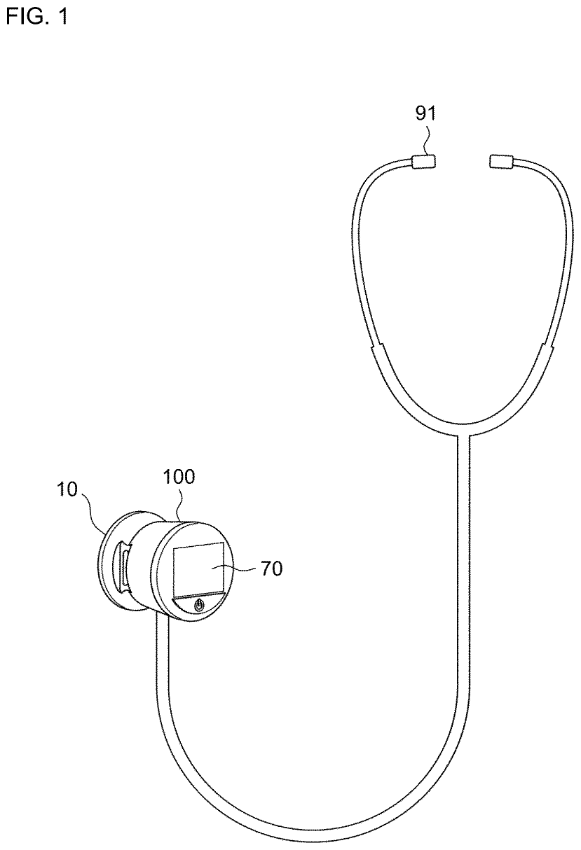

FIG. 1 is a schematic diagram illustrating an example of an auscultation device 100 according to a first embodiment. The auscultation device 100 includes a body sound acquisition unit 10 that detects biological signals as sounds. The body sound acquisition unit 10 is, for example, a microphone or a vibration sensor. A user, such as a physician or a healthcare professional, brings the body sound acquisition unit 10 into contact with the skin of a patient so that the body sound acquisition unit 10 can detect body sounds and output the detected body sounds as electric signals.

The user can listen to sounds generated by the auscultation device 100 based on the body sounds via an audio output device 91, such as earphones. In the example illustrated in FIG. 1, the audio output device 91 is wired to the auscultation device 100 via an interface, such as an earphone jack. However, the audio output device 91 may be wirelessly connected to the auscultation device 100 for communication.

Furthermore, the auscultation device 100 includes a display monitor 70 that displays information based on body sounds obtained by the body sound acquisition unit 10. The user can understand the conditions of a patient by viewing the information displayed on the display monitor 70.

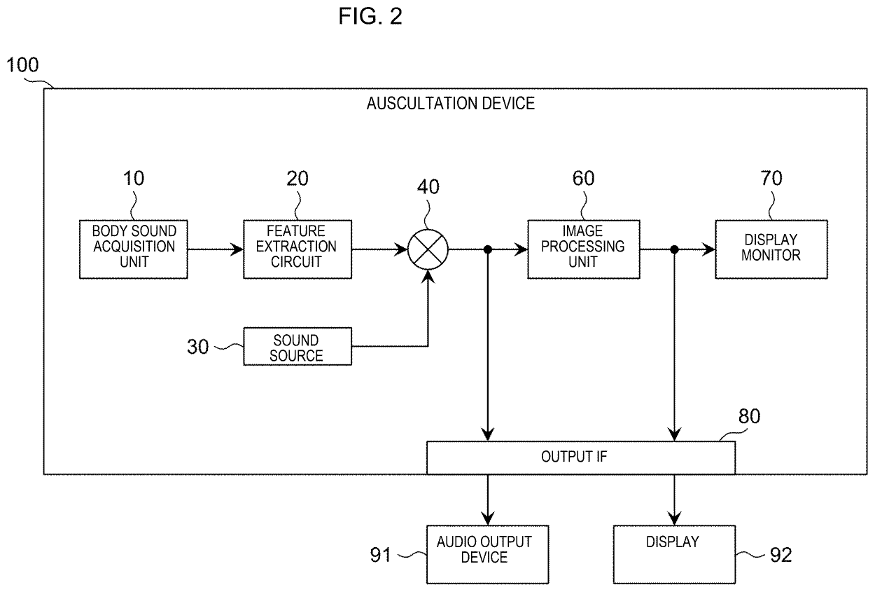

FIG. 2 is a block diagram illustrating an example of a functional configuration of the auscultation device 100 in FIG. 1. As described above, the auscultation device 100 includes the body sound acquisition unit 10 and the display monitor 70. The auscultation device 100 further includes a feature extraction circuit 20, a sound source 30, a mixer circuit 40, an image processing unit 60, and an output interface 80.

The feature extraction circuit 20 extracts, from a body sound obtained by the body sound acquisition unit 10, a feature signal representing the feature of the body sound. The feature signal represents a feature of, for example, a high-frequency sound, such as a wheezing sound or a whistling sound, a low-frequency sound, such as a blood flow sound, a specific frequency component representing an asthma attack sound, or other noise. Here, a high-frequency sound refers to a sound having a frequency higher than a predetermined first frequency, and a low-frequency sound refers to a sound having a frequency lower than a predetermined second frequency. The first frequency may be the same as or different from the second frequency.

Feature signals may also indicate the frequency of occurrence of asthma attacks, the frequency of occurrence of whistling sounds, and the frequency of occurrence of other noise. In addition to, or instead of, the above examples, feature signals may indicate the degree of aortic stenosis, the degree of regurgitation, and the frequency of occurrence of palpitations. In addition to, or instead of, the above examples, feature signals may indicate the degree of hypersensitivity inflammation, the degree of interstitial inflammation, and the frequency of occurrence of fine crackles.

The sound source 30 is a sound source device that outputs a sound source signal with a predetermined frequency. The predetermined frequency is, for example, but not limited to, a frequency less than or equal to 1500 Hz, such as 600 Hz, that can be easily heard even by an elderly person.

The mixer circuit 40 is a mixing circuit, or a frequency mixer, that mixes two inputs and outputs a mixed result.

The image processing unit 60 generates image data based on a signal received from the mixer circuit 40. For example, the image processing unit 60 is implemented by a processor, such as a central processing unit (CPU) or a graphics processing unit (GPU). The image processing unit 60 may be implemented by one or more processors. The image processing unit 60 operates in accordance with instructions of a program stored in a storage device, such as a ROM, and thereby implements the function as described above. Also, the image processing unit 60 may include a memory, such as a RAM, that functions as a working area for the processor.

The display monitor 70 is a display device, such as a liquid crystal display or an organic EL display, that is capable of displaying information. The display monitor 70 displays an image represented by image data generated by the image processing unit 60.

The output interface 80 connects the auscultation device 100 to output devices, such as the audio output device 91 and a display 92, to enable the auscultation device 100 to output signals or information to the output devices. The output interface 80 may be a communication circuit that performs data communication according to an existing wired communication standard or wireless communication standard.

### 1-2. Operation

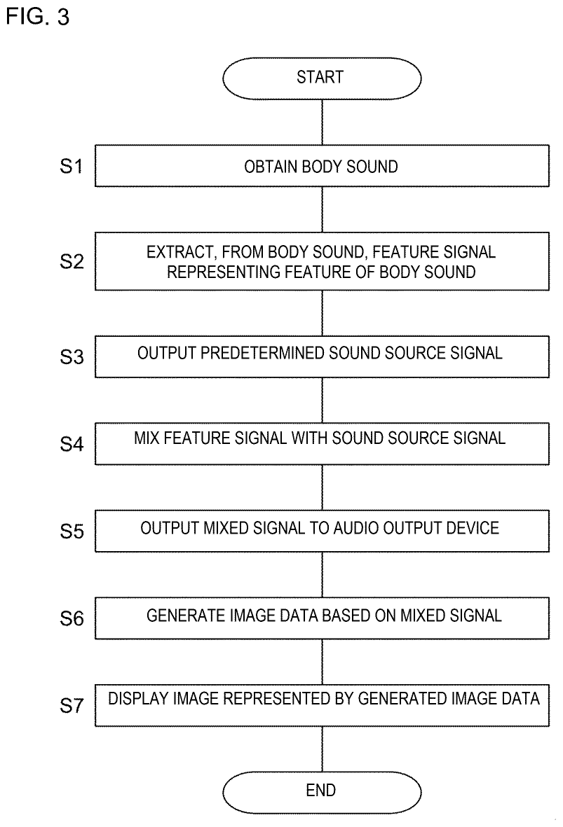

An operation of the auscultation device 100 with the above configuration is described below. FIG. 3 is a flowchart illustrating an example of an operation of the auscultation device 100 according to the present embodiment.

First, the body sound acquisition unit 10 obtains a body sound (S1). For example, the body sound acquisition unit 10 detects a body sound by using a microphone or a vibration sensor and outputs the detected body sound as an electric signal.

Next, the feature extraction circuit 20 extracts, from the body sound obtained by the body sound acquisition unit 10, a feature signal that represents the feature of the body sound (S2).

The sound source 30 outputs a sound source signal with a predetermined frequency (S3). Step S3 may be performed before step S1 or between steps S1 and S2, unlike the example illustrated in FIG. 3.

The mixer circuit 40 generates a mixed signal by mixing the feature signal extracted by the feature extraction circuit 20 with the sound source signal outputted from the sound source 30 (S4).

The auscultation device 100 outputs the mixed signal generated by the mixer circuit 40 to the audio output device 91 via the output interface 80 (S5). The auscultation device 100 includes, for example, a processor, such as a CPU, or a control unit including a processor, and controls step S5 using the processor.

Sounds outputted from the audio output device 91 are described below. Body sounds, which a user, such as a physician or a healthcare professional, listens to by using a related-art stethoscope, may include various sounds, such as a high-frequency sound like a wheezing sound, and a low-frequency, low volume sound like a blood flow sound. These sounds may include sounds that are difficult to hear for some users. For example, elderly users may have difficulty in hearing high-frequency sounds, such as a wheezing sound and a whistling sound. When a body sound includes a predetermined feature, the auscultation device 100 of the present embodiment performs steps S2 to S4 to adjust an output sound according to the feature. This makes it easier for the user to recognize that a body sound includes a predetermined feature.

When no signal is inputted to the mixer circuit 40 from the feature extraction circuit 20, the mixer circuit 40 outputs the sound source signal (e.g., a tone with a frequency of 600 Hz) inputted from the sound source 30.

When receiving a feature signal from the feature extraction circuit 20, the mixer circuit 40 mixes the feature signal with the sound source signal and outputs the mixed result. For example, when receiving a feature signal from the feature extraction circuit 20, the mixer circuit 40 outputs a signal that is obtained by changing at least one of the frequency and the amplitude of the sound source signal based on a comparison with the feature signal according to the waveform of the feature signal. For example, the mixer circuit 40 increases the frequency of an output signal according to the level of a high-frequency sound, such as a wheezing sound or a whistling sound. In addition to or instead of this example, the mixer circuit 40 may decrease the amplitude of an output signal or the volume of an output sound when a (non-asthmatic) low-frequency sound component, which is unrelated to the feature of asthma, in the feature signal is greater than or equal to a predetermined value. In addition to or instead of this example, when a high-frequency sound, such as a wheezing sound or a whistling sound, is inputted, the mixer circuit 40 may shift the frequency of the input signal to fall within a range that is easily audible by the user.

The image processing unit 60 generates image data based on the mixed signal generated at step S4 by the mixer circuit 40 (S6). The display monitor 70 displays an image represented by the image data generated by the image processing unit 60 (S7).

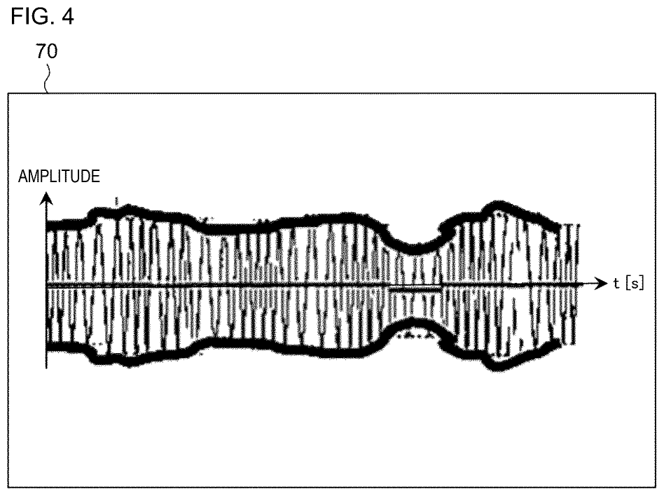

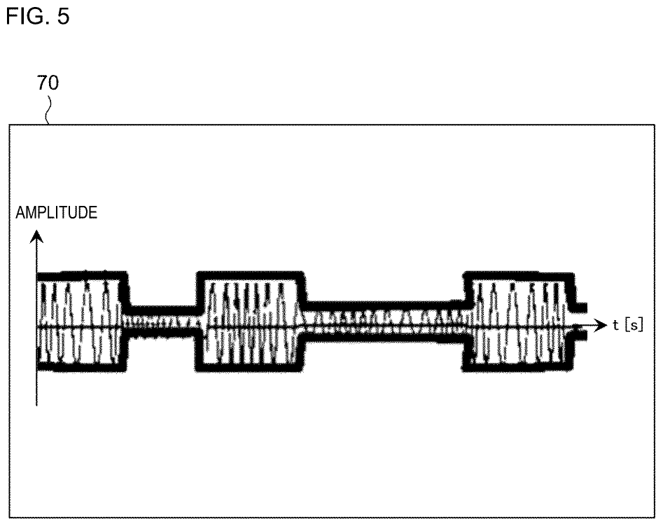

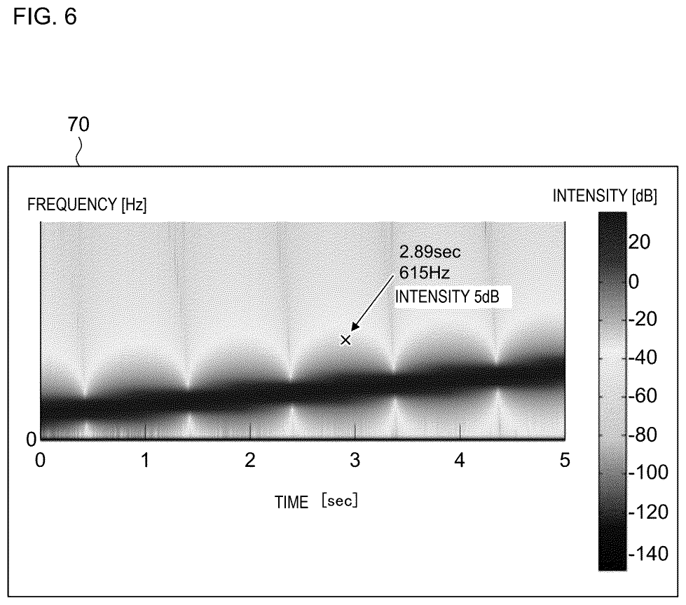

FIGS. 4 to 6 are schematic diagrams showing examples of screens displayed on the display monitor 70 at step S7. The image data generated based on the mixed signal at step S6 represents, for example, a time-amplitude graph of the mixed signal as shown in each of FIGS. 4 and 5 or a spectrogram image as shown in FIG. 6. Thus, the display monitor 70 visually displays the feature of a body sound. The user can identify a body sound and thereby determine the condition of a patient by viewing information displayed on the display monitor 70.

In addition to or instead of step S7, the auscultation device 100 may output the image data generated by the image processing unit 60 via the output interface 80 to an external device, such as the display 92 or an external computer.

The auscultation device 100 only needs to output at least one of the mixed signal generated by the mixer circuit 40 or the information based on the mixed signal. Therefore, the auscultation device 100 may be configured to perform only steps S1 to S5 or steps S1 to S4, S6, and S7 instead of the entire process shown in FIG. 3.

The auscultation device 100 can also detect asthma based on the matching degree between a mixed signal generated by the mixer circuit 40 and a signal pattern representing the feature of asthma.

### 1-3. Summary

As described above, the auscultation device 100 according to the present embodiment includes the body sound acquisition unit 10 that obtains a body sound, the feature extraction circuit 20, the sound source 30 that outputs a predetermined sound source signal, the mixer circuit 40, and the output interface 80, which is an example of an output unit. The feature extraction circuit 20 extracts, from a body sound obtained by the body sound acquisition unit 10, a feature signal representing the feature of the body sound. The mixer circuit 40 mixes the feature signal extracted by the feature extraction circuit 20 with the sound source signal outputted from the sound source 30. The output interface 80 outputs at least one of a signal outputted from the mixer circuit 40 and information that is based on the signal.

The above configuration makes it easier for the user to recognize a body sound of a subject and thereby recognize the condition of the subject based on a signal or information outputted from the auscultation device 100.

The auscultation device 100 according to the present embodiment may further include the image processing unit 60. Based on a signal outputted from the mixer circuit 40, the image processing unit 60 generates image data for visually displaying the feature of a body sound. In this case, the output interface 80 outputs the image data as the information based on the signal. The auscultation device 100 may further include the display monitor 70 that displays the image data.

This configuration enables the user to visually recognize a body sound of a subject and thereby recognize the condition of the subject based on the image data.

### 2. Second Embodiment

An auscultation device according to a second embodiment is described below. In the following descriptions of the auscultation device according to the present embodiment, the same reference numbers as those assigned to the steps and components of the auscultation device according to the first embodiment are assigned to the corresponding steps and components, and descriptions of those steps and components may be omitted.

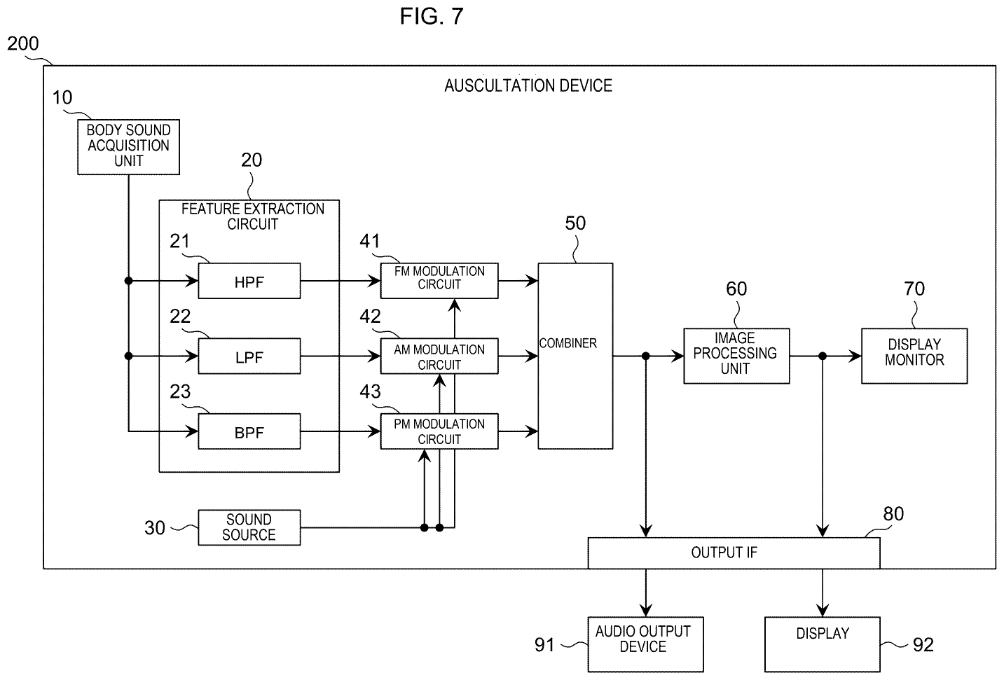

FIG. 7 is a block diagram illustrating an example of a configuration of an auscultation device 200 according to the second embodiment. Compared to the auscultation device 100 in FIG. 1, the auscultation device 200 additionally includes a combiner 50. The auscultation device 200 also includes, in place of the mixer circuit 40, a frequency modulation circuit (hereafter referred to as “FM modulation circuit”) 41, an amplitude modulation circuit (hereafter referred to as “AM modulation circuit”) 42, and a phase modulation circuit (hereafter referred to as “PM modulation circuit”) 43.

In the present embodiment, the feature extraction circuit 20 includes a high pass filter (HPF) 21, a low pass filter (LPF) 22, and a band pass filter (BPF) 23.

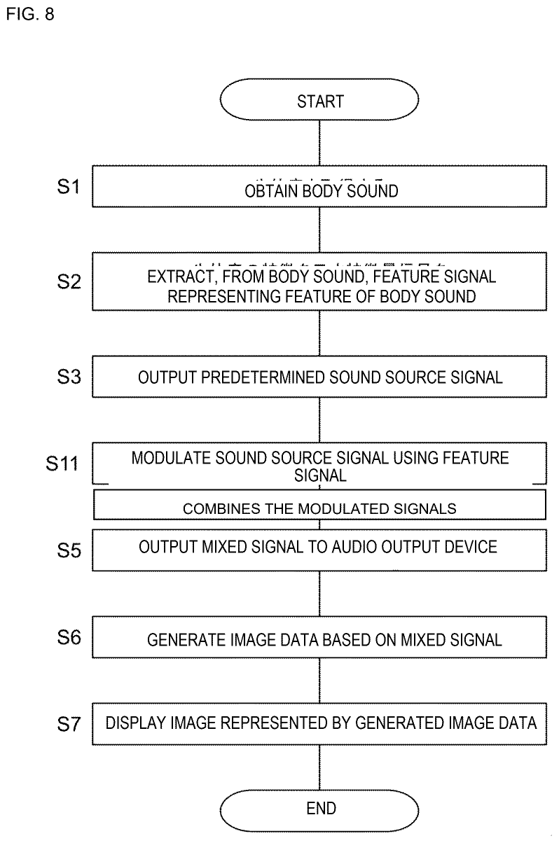

FIG. 8 is a flowchart illustrating an example of an operation of the auscultation device 200 according to the present embodiment. Compared to the flowchart of the first embodiment in FIG. 3, the flowchart in FIG. 8 includes step S11 instead of step S4.

Referring to FIGS. 7 and 8, a body sound obtained by the body sound acquisition unit 10 is inputted to the HPF 21, the LPF 22, and the BPF 23. Each of the HPF 21, the LPF 22, and the BPF 23 extracts a feature signal representing a feature of the body sound (S2).

For example, the auscultation device 200 according to the present embodiment is usable to extract features related to asthma of a patient. The HPF 21 can extract a high-frequency component, such as a whistling sound related to asthma, from a body sound. The cutoff frequency of the HPF 21 is, for example, but not limited to, from 1 kHz to 10 KHz. The LPF 22 can extract, from a body sound, a low-frequency sound (hereafter may be referred to as “non-asthmatic low-frequency sound”), such as a blood flow sound or noise, that does not show the feature of asthma. The cutoff frequency of the LPF 22 is, for example, but not limited to, from 10 Hz to 1 kHz. The BPF 23 can extract a specific frequency component representing the feature of an asthma attack sound from a body sound. The pass band width of the BPF 23 is, for example, but not limited to, from 1 kHz to 3 kHz.

The FM modulation circuit 41, the AM modulation circuit 42, and the PM modulation circuit 43 modulate a sound source signal outputted from the sound source 30 by using the input feature signals (S11). The sound source signal is a carrier wave to be modulated. In the example illustrated in FIG. 7, the FM modulation circuit 41 modulates the sound source signal by using a high-frequency sound feature signal outputted from the HPF 21, the AM modulation circuit 42 modulates the sound source signal by using a low-frequency sound feature signal outputted from the LPF 22, and the PM modulation circuit 43 modulates the sound source signal by using a signal outputted from the BPF 23.

The combiner 50 combines the modulated signals outputted from the FM modulation circuit 41, the AM modulation circuit 42, and the PM modulation circuit 43. For example, the combiner 50 adds the modulated signals outputted from the FM modulation circuit 41, the AM modulation circuit 42, and the PM modulation circuit 43.

The auscultation device 200 outputs the combined signal outputted from the combiner 50 to the audio output device 91 via the output interface 80 (S5). The image processing unit 60 generates image data based on the combined signal outputted from the combiner 50 (S6). The display monitor 70 displays an image represented by the image data generated by the image processing unit 60 (S7).

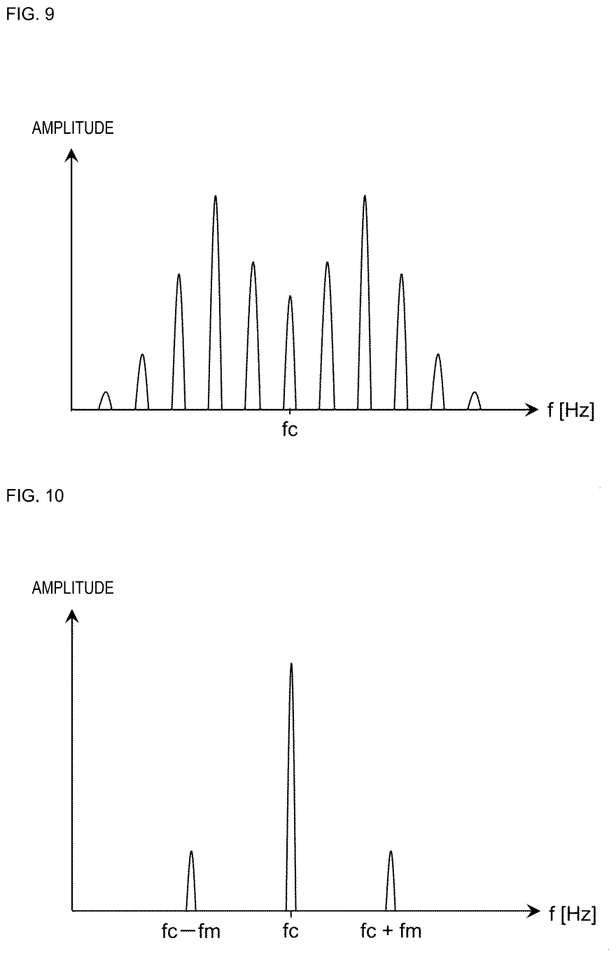

As with the first embodiment, the image data generated at step S6 in the present embodiment may represent a time-amplitude graph (see FIGS. 4 and 5) and/or a spectrogram image (see FIG. 6). The image data generated at step S6 in the present embodiment may represent an image showing a spectrum (a frequency-amplitude graph) as shown in FIG. 9 or FIG. 10. In FIGS. 9 and 10, fc indicates the frequency of a carrier wave (sound source signal). In FIG. 10, fm indicates the frequency of a sound source signal, which is a carrier wave.

In the example described above, the auscultation device 200 includes three modulation circuits. However, the auscultation device only needs to include one or more modulation circuits and may include, for example, only one FM modulation circuit 41. When the auscultation device 200 includes only one modulation circuit, the auscultation device 200 need not include the combiner 50.

As described above, the auscultation device 200 of the present embodiment includes the body sound acquisition unit 10 that obtains a body sound, the feature extraction circuit 20, the sound source 30 that outputs a predetermined sound source signal, the modulation circuit, and the output interface 80. The feature extraction circuit 20 extracts, from the body sound obtained by the body sound acquisition unit 10, a feature signal representing the feature of the body sound. The modulation circuit modulates the sound source signal by using the feature signal. The output interface 80 outputs at least one of a signal outputted from the combiner 50 and information that is based on the signal.

The above configuration makes it easier for the user to recognize a body sound of a subject and thereby recognize the condition of the subject based on a signal or information outputted from the auscultation device 200.

The feature extraction circuit 20 may extract, from the body sound, a feature signal representing the feature of asthma.

This configuration makes it easier for the user to recognize information related to asthma.

The feature extraction circuit 20 may include the HPF 21 (an example of a first extraction circuit) that extracts, from the body sound, a first signal with a frequency higher than a predetermined frequency, and the BPF 23 (an example of a second extraction circuit) that extracts, from the body sound, a second signal representing a specific frequency component that represents an asthma attack sound. The modulation circuit may include a first modulator that modulates the sound source signal by using the first signal and a second modulator that modulates the sound source signal by using the second signal. For example, the first modulator includes the FM modulation circuit 41, and the second modulator includes the PM modulation circuit 43.

The feature extraction circuit 20 may further include the LPF 22 (an example of a third extraction circuit) that extracts, from the body sound, a third signal representing noise. In this case, the modulation circuit may further include a third modulator that modulates the sound source signal by using the third signal. For example, the third modulator includes the PM modulation circuit 43.

The auscultation device 200 of the present embodiment may further include the combiner 50 that combines a first modulated signal outputted from the first modulator with a second modulated signal outputted from the second modulator.

The auscultation device 200 of the present embodiment may further include the image processing unit 60 that generates image data for visually displaying the feature of the body sound based on a signal outputted from the modulation circuit. In this case, the output interface 80 outputs the image data as the information based on the signal. The auscultation device 200 may further include the display monitor 70 that displays the image data.

This configuration enables the user to visually recognize a body sound of a subject and thereby recognize the condition of the subject based on the image data.

The output interface 80 may output the signal, which is outputted from the modulation circuit, as a sound.

This configuration enables the user to recognize a body sound of a subject and thereby recognize the condition of the subject based on the sound.

### 3. Third Embodiment

An auscultation device according to a third embodiment is described below. In the following descriptions of the auscultation device according to the present embodiment, the same reference numbers as those assigned to the steps and components of the auscultation devices according to the first embodiment and the second embodiment are assigned to the corresponding steps and components, and descriptions of those steps and components may be omitted.

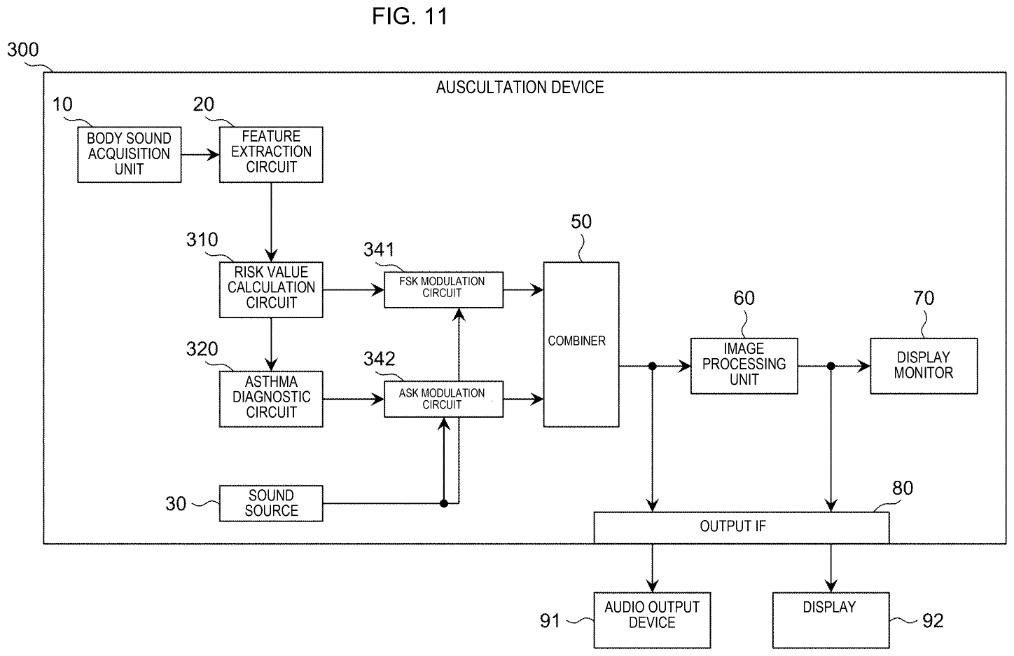

FIG. 11 is a block diagram illustrating an example of a configuration of an auscultation device 300 according to the third embodiment. The auscultation device 300 includes a body sound acquisition unit 10, a feature extraction circuit 20, a sound source 30, a combiner 50, an image processing unit 60, a display monitor 70, and an output interface 80. Because these components are substantially the same as the corresponding components of the auscultation devices according to the first embodiment and the second embodiment, detailed descriptions of these components are omitted.

The auscultation device 300 further includes a risk value calculation circuit 310, an asthma diagnostic circuit 320, a frequency shift keying modulation circuit (hereafter referred to as “FSK modulation circuit”) 341, and an amplitude shift keying modulation circuit (hereafter referred to as “ASK modulation circuit”) 342.

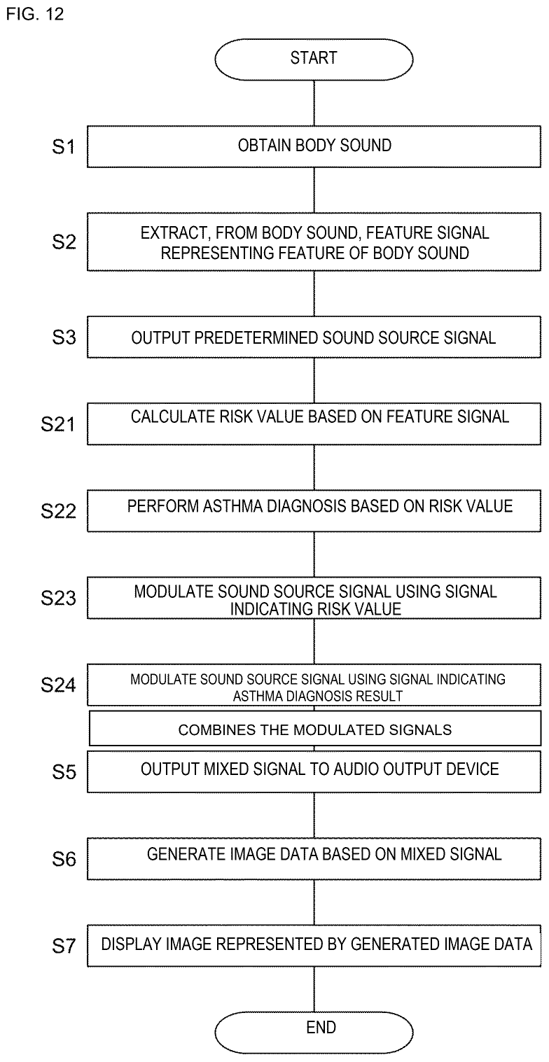

FIG. 12 is a flowchart illustrating an example of an operation of the auscultation device 300 according to the present embodiment. First, the body sound acquisition unit 10 obtains a body sound (S1). The feature extraction circuit 20 extracts, from the body sound, a feature signal representing the feature of the body sound obtained by the body sound acquisition unit 10 (S2). The sound source 30 outputs a sound source signal with a predetermined frequency (S3).

The risk value calculation circuit 310 calculates a risk value based on the feature signal extracted by the feature extraction circuit 20 (S21). For example, the risk value calculation circuit 310 performs fast Fourier transform (FFT) on the feature signal and calculates the risk value based on auscultation observation data that is the result of the FFT processing.

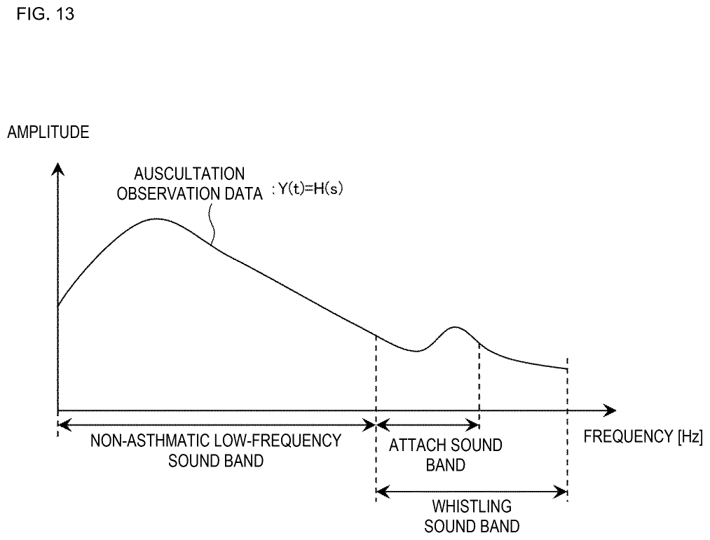

FIG. 13 is a graph for describing an example of a method of calculating a risk value. The graph in FIG. 13 shows auscultation observation data, which is the result of the FFT processing performed by the risk value calculation circuit 310. The risk value calculation circuit 310 calculates at least one of attach sound energy E_S, non-asthmatic low-frequency sound energy E_L, whistling sound energy E_H, and a disease risk value based on the result of the FFT processing.

The attach sound energy E_S represents the energy of a specific frequency component that represents the feature of the asthma attack sound. The attach sound energy E_S is, for example, the percentage of energy in an attach sound band relative to the total energy of the auscultation observation data. The attach sound band is, for example, between 1 kHz and 3 kHz.

The non-asthmatic low-frequency sound energy E_L represents the energy of a (non-asthmatic) low-frequency sound component that is unrelated to the feature of asthma. The non-asthmatic low-frequency sound energy E_L is, for example, the percentage of energy in a non-asthmatic low-frequency sound band relative to the total energy of the auscultation observation data. The non-asthmatic low-frequency sound band is, for example, between 10 Hz and 1 KHz.

The whistling sound energy E_H represents the energy of a whistling sound component that represents the feature of asthma. The whistling sound energy E_H is, for example, the percentage of energy in a whistling sound band relative to the total energy of the auscultation observation data. The whistling sound band is, for example, between 1 kHz and 10 KHz.

The disease risk value is calculated based on the attach sound energy E_S, the non-asthmatic low-frequency sound energy E_L, and the whistling sound energy E_H and indicates a risk of asthma. The disease risk value is, for example, the sum of E_H/E_L and E_S/E_L.

Returning to FIG. 12, the asthma diagnostic circuit 320 performs an asthma diagnosis based on the risk value calculated by the risk value calculation circuit 310 (S22). For example, the asthma diagnostic circuit 320 outputs a digital value “1” or “H” indicating asthma when the risk value is greater than or equal to a predetermined threshold and outputs a digital value “0” or “L” indicating that there is no symptom of asthma when the risk value is less than the predetermined threshold.

The auscultation device 300 may output a signal indicating the risk value calculated by the risk value calculation circuit 310 or output a signal indicating the diagnosis result of the asthma diagnostic circuit 320. For example, the FSK modulation circuit 341 modulates the sound source signal outputted from the sound source 30 by using the signal indicating the risk value (S23), and the ASK modulation circuit 342 modulates the sound source signal by using the signal indicating the diagnosis result (S24). The combiner 50 combines the modulated signals outputted from the FSK modulation circuit 341 and the ASK modulation circuit 342. For example, the combiner 50 adds the modulated signals.

The auscultation device 300 outputs the combined signal, which is outputted from the combiner 50, to the audio output device 91 via the output interface 80 (S5). The image processing unit 60 generates image data based on the combined signal outputted from the combiner 50 (S6). The display monitor 70 displays an image represented by the image data generated by the image processing unit 60 (S7).

The auscultation device 300 according to the present embodiment employs different modulation schemes (ASK and FSK) for respective features and outputs a sound obtained by combining modulated signals. The user can determine the magnitudes of respective features based on the output sound. When image data is generated based on the combined signal, the user can determine the magnitudes of respective features based on the image data.

For example, in the present embodiment, a sound only the frequency of which varies may be outputted for a case in which the risk value is high but the diagnosis result is L (non-asthmatic), and a sound with a greater frequency variation and a higher volume may be outputted for a case in which the risk value is high and the diagnosis result is H (asthma). This enables the user to instantly identify multiple parameters from a single sound.

In the above example, ASK modulation is performed on a signal indicating the diagnosis result of the asthma diagnostic circuit 320. However, the auscultation device 300 may include a pulse modulation circuit in place of the ASK modulation circuit 342 and perform pulse modulation on a signal indicating the diagnosis result.

Also, in the example described above, the auscultation device 300 includes two modulation circuits. However, the auscultation device may include any number of modulation circuits and may be configured to include no modulation circuit. When the auscultation device 300 includes only one modulation circuit or includes no modulation circuit, the auscultation device 300 need not include the combiner 50.

### 4. Fourth Embodiment

In each of the embodiments described above, the auscultation device also performs processes, such as a feature extraction process, other than a body sound acquisition process. However, the auscultation device may be configured to perform only a body sound acquisition process, and other processes may be performed by another device. An auscultation system according to a fourth embodiment is described below as an example of this configuration.

In the descriptions of an auscultation system according to the present embodiment, the same reference numbers as those assigned to the steps and components of the auscultation device 100 according to the first embodiment are assigned to the corresponding steps and components, and descriptions of those steps and components may be omitted. The present embodiment may be combined with the second embodiment or the third embodiment instead of the first embodiment.

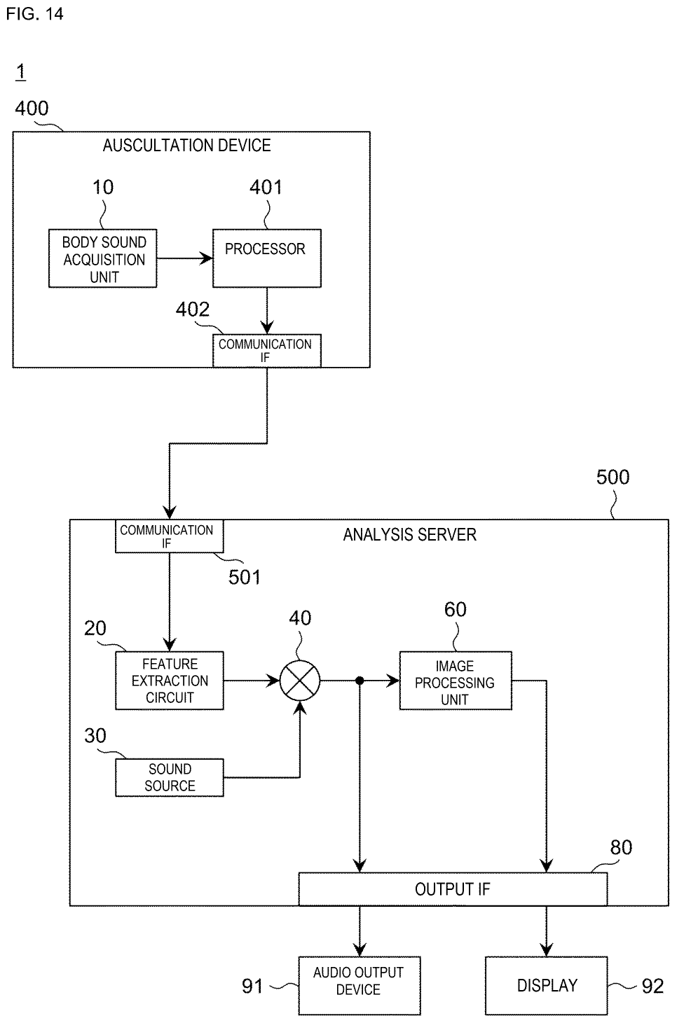

FIG. 14 is a block diagram illustrating an example of a configuration of an auscultation system 1 according to the fourth embodiment. The auscultation system 1 includes an auscultation device 400 and an analysis server 500.

The auscultation device 400 includes a body sound acquisition unit 10, a processor 401, and a communication interface 402. The processor 401 includes an arithmetic circuit, such as a CPU. The communication interface 402 includes a communication circuit that performs data communication according to an existing wired communication standard or wireless communication standard.

The auscultation device 400 is used, for example, by a patient. The patient places the auscultation device 400 on their chest to obtain body sound data and transmits the obtained body sound data to the analysis server 500.

The analysis server 500 includes a feature extraction circuit 20, a sound source 30, a mixer circuit 40, an image processing unit 60, an output interface 80, and a communication interface 501. Also, the analysis server 500 includes, for example, a processor, such as a CPU, or a control unit including a processor, and implements functions described below using the processor.

The analysis server 500 receives the body sound data transmitted from the auscultation device 400 via the communication interface 501.

The feature extraction circuit 20 extracts, from the received body sound, a feature signal representing the feature of the body sound. The mixer circuit 40 generates a mixed signal by mixing the feature signal and a sound source signal outputted from the sound source 30. The analysis server 500 outputs the mixed signal generated by the mixer circuit 40 to the audio output device 91 via the output interface 80. The audio output device 91 is a device, such as earphones or a PC, used by a user, such as a physician or a healthcare professional, and is connected to the analysis server 500 via, for example, a network. This enables the user to remotely recognize that a given feature is included in the body sound obtained by the auscultation device 400.

The image processing unit 60 generates image data based on the mixed signal generated by the mixer circuit 40. The analysis server 500 outputs the image data generated by the image processing unit 60, via the output interface 80, to the display 92 that is usable by the user, such as a physician or a healthcare professional. The user can remotely recognize the body sound and thereby recognize the condition of the patient by viewing information displayed on the display 92.

In the example described above, the analysis server 500 includes the feature extraction circuit 20, the sound source 30, the mixer circuit 40, and the image processing unit 60. However, the functions of these components are not necessarily implemented by one analysis server 500 but may be implemented by multiple devices that are connected to each other.

### 5. Other Embodiments

The above-described embodiments are examples of technologies provided by the present disclosure. However, the technologies provided by the present disclosure are not limited to the above embodiments, and modifications, substitutions, additions, and omissions may be applied to the above embodiments as necessary. Also, a new embodiment may be made by combining components described in the above embodiments. Variations are described below as other embodiments.

### 5-1. First Variation

In the example of the second embodiment, as illustrated in FIG. 7, the auscultation device 200 includes analog modulation circuits. Alternatively, an auscultation device according to the present disclosure may include digital modulation circuits in place of analog modulation circuits. Compared to FIG. 7, such an auscultation device may include, for example, an FSK modulation circuit in place of the FM modulation circuit 41, an ASK modulation circuit in place of the AM modulation circuit 42, and a PSK modulation circuit in place of the PM modulation circuit 43.

### 5-2. Second Variation

In the examples of the above embodiments, the audio output device 91 receives a signal outputted from the mixer circuit 40 or a signal outputted from a modulation circuit. Alternatively, the audio output device 91 may receive a body sound obtained by the body sound acquisition unit 10.

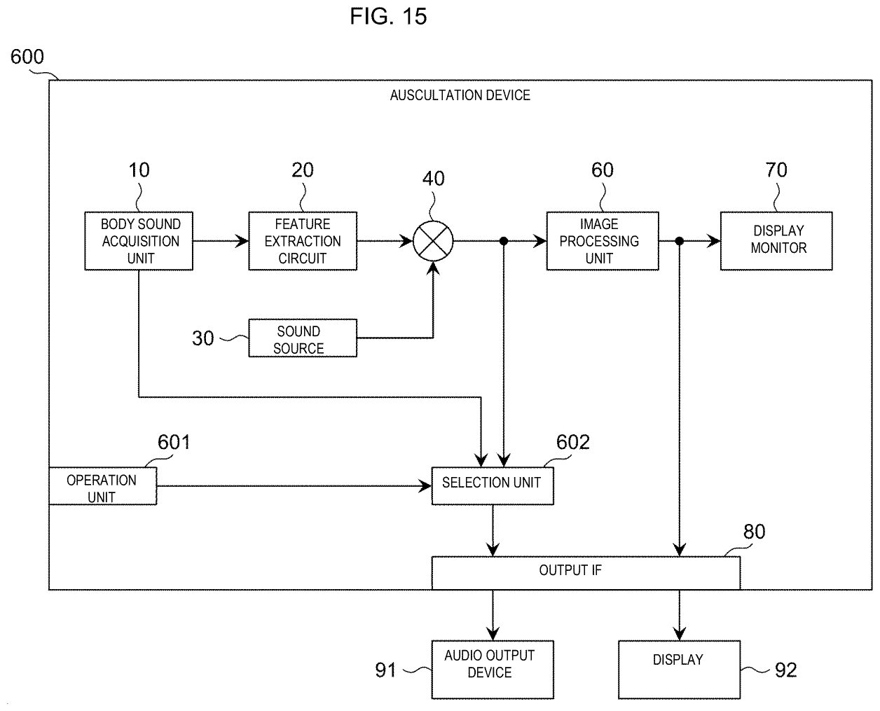

FIG. 15 is a block diagram illustrating an example of a configuration of an auscultation device 600 according to a second variation. Compared to the auscultation device 100 in FIG. 1, the auscultation device 600 further includes an operation unit 601 and a selection unit 602.

The operation unit 601 is, for example, a button provided on the exterior of the auscultation device 600. The operation unit 601 transmits a switching signal to the selection unit 602 in response to a user operation, such as the pressing of the button.

According to the switching signal, the selection unit 602 switches signals outputting to the audio output device 91. For example, according to the switching signal, the selection unit 602 selects one of a body sound obtained by the body sound acquisition unit 10 and a signal outputted from the mixer circuit 40 for outputting to the audio output device 91. Also, the selection unit 602 may output both of the body sound obtained by the body sound acquisition unit 10 and the signal outputted from the mixer circuit 40 to the audio output device 91 according to the switching signal.

### 6. Aspects

Aspects of the present disclosure are described below.

<Aspect 1> An auscultation system includes a body sound acquisition unit that obtains a body sound; a feature extraction circuit that extracts, from the body sound, a feature signal representing the feature of the body sound; a sound source that outputs a predetermined sound source signal; a mixer circuit that mixes the feature signal with the sound source signal; and an output unit that outputs, for visual display, at least one of a signal outputted from the mixer circuit and information that is based on the signal.

<Aspect 2> The auscultation system described in Aspect 1 further includes an image processing unit that generates image data for visually displaying the feature of the body sound based on the signal outputted from the mixer circuit. The output unit outputs the image data as the information.

<Aspect 3> The auscultation system described in Aspect 1 or 2 further includes an image processing unit that generates image data for visually displaying the feature of the body sound based on the signal outputted from the mixer circuit; and a display unit that displays the image data.

<Aspect 4> An auscultation system includes a body sound acquisition unit that obtains a body sound; a feature extraction circuit that extracts a feature signal from the body sound; a sound source that outputs a predetermined sound source signal; a modulation circuit that modulates the sound source signal by using the feature signal; and an output unit that outputs at least one of a signal outputted from the modulation circuit and information that is based on the signal.

<Aspect 5> In the auscultation system described in Aspect 4, the feature extraction circuit extracts, from the body sound, a signal representing a feature of asthma as the feature signal.

<Aspect 6> In the auscultation system described in Aspect 5, the feature extraction circuit includes a first extraction circuit that extracts, from the body sound, a first signal representing a feature of a whistling sound and a second extraction circuit that extracts, from the body sound, a second signal representing a specific frequency component that represents an asthma attack sound; and the modulation circuit includes a first modulator that modulates the sound source signal by using the first signal and a second modulator that modulates the sound source signal by using the second signal.

<Aspect 7> In the auscultation system described in Aspect 6, the feature extraction circuit further includes a third extraction circuit that extracts a third signal representing noise from the body sound, and the modulation circuit further includes a third modulator that modulates the sound source signal by using the third signal.

<Aspect 8> The auscultation system described in Aspect 6 or 7 further includes a combiner that combines a first modulated signal outputted from the first modulator with a second modulated signal outputted from the second modulator.

<Aspect 9> In the auscultation system described in any one of Aspects 6 to 8, the first modulator includes an FM modulation circuit, and the second modulator includes a PM modulation circuit.

<Aspect 10> The auscultation system described in any one of Aspects 4 to 9 further includes an image processing unit that generates image data for visually displaying the feature of the body sound based on the signal outputted from the modulation circuit. The output unit outputs the image data as the information.

<Aspect 11> The auscultation system described in any one of Aspects 4 to 10 further includes an image processing unit that generates image data for visually displaying the feature of the body sound based on the signal outputted from the modulation circuit and a display unit that displays the image data.

<Aspect 12> In the auscultation system described in any one of Aspects 4 to 11, the output unit outputs the signal, which is outputted from the modulation circuit, as a sound.

<Aspect 13> In the auscultation system described in Aspect 12, the output unit is configured to be able to output the body sound obtained by the body sound acquisition unit, and the auscultation system further comprises a selection unit that selects whether to output the sound or the body sound from the output unit.

The present disclosure is applicable to an auscultation device and an auscultation system.

What is claimed is:

1. An auscultation system comprising:
a body sound sensor configured to obtain a body sound;
a feature extraction circuit configured to extract, from the body sound, a feature signal representing a feature of the body sound;
a sound source configured to output a predetermined sound source signal;
a mixer circuit configured to mix the feature signal with the sound source signal; and
an output configured to visually display a signal output from the mixer circuit or information that is based on the signal.

2. The auscultation system according to claim 1, further comprising:
an image processor configured to generate image data for visually displaying the feature of the body sound based on the signal output from the mixer circuit,
wherein the output visually displays the image data as the information.

3. The auscultation system according to claim 1, further comprising:
an image processor configured to generate image data for visually displaying the feature of the body sound based on the signal output from the mixer circuit; and
a display configured to display the image data.

4. An auscultation system comprising:
a body sound sensor configured to obtain a body sound;
a feature extraction circuit configured to extract a feature signal from the body sound;
a sound source configured to output a predetermined sound source signal;
a modulation circuit configured to modulate the sound source signal by using the feature signal; and
an output configured to output a signal that is output from the modulation circuit or information that is based on the signal.

5. The auscultation system according to claim 4, wherein the feature extraction circuit is configured to extract, from the body sound, a signal representing a feature of asthma as the feature signal.

6. The auscultation system according to claim 5,
wherein the feature extraction circuit comprises:
a first extraction circuit configured to extract, from the body sound, a first signal representing a feature of a whistling sound, and
a second extraction circuit configured to extract, from the body sound, a second signal representing a specific frequency component that represents an asthma attack sound; and
wherein the modulation circuit comprises:
a first modulator configured to modulate the sound source signal by using the first signal, and
a second modulator configured to modulate the sound source signal by using the second signal.

7. The auscultation system according to claim 6,
wherein the feature extraction circuit further comprises a third extraction circuit configured to extract a third signal representing noise from the body sound; and
wherein the modulation circuit further comprises a third modulator configured to modulate the sound source signal by using the third signal.

8. The auscultation system according to claim 6, further comprising:
a combiner configured to combine a first modulated signal output from the first modulator with a second modulated signal output from the second modulator.

9. The auscultation system according to claim 6,
wherein the first modulator comprises a frequency modulation (FM) circuit, and
wherein the second modulator comprises a phase modulation (PM) circuit.

10. The auscultation system according to claim 4, further comprising:
an image processor configured to generate image data for visually displaying the feature of the body sound based on the signal output from the modulation circuit,
wherein the output is configured to output the image data as the information.

11. The auscultation system according to claim 4, further comprising:
an image processor configured to generate image data for visually displaying the feature of the body sound based on the signal output from the modulation circuit; and
a display configured to display the image data.

12. The auscultation system according to claim 4, wherein the output is configured to output the signal from the modulation circuit as a sound.

13. The auscultation system according to claim 12,
wherein the output is configured to output the body sound obtained by the body sound sensor, and
wherein the auscultation system is further configured to select whether to output the sound or the body sound from the output.
